(19) United States
(12) Patent Application Publication
(10) Pub. No.: US 2021/0395693 A1
(43) Pub. Date: Dec. 23, 2021

(54) METHOD AND APPARATUS FOR MESENCHYMAL STEM CELLS ISOLATION AND PURIFICATION

(71) Applicants: Guy MARTI, Montigny-sur-Loing (FR); STEMSELECT, Moret-Loing-et-Orvanne (FR)

(72) Inventors: Guy MARTI, Montigny-sur-Loing (FR); Aurelien Markus MARTI, Veneux-les-Sablons (FR)

(73) Assignees: Guy MARTI, Montigny-sur-Loing (FR); STEMSELECT, Moret-Loing-et-Orvanne (FR)

(21) Appl. No.: 17/285,596

(22) PCT Filed: Oct. 25, 2019
(86) PCT No.: PCT/EP2019/079240

§ 371 Date: Apr. 15, 2021

(30) Foreign Application Priority Data
Oct. 26, 2018 (EP) 18306401.3

(51) Int. Cl. C12N 5/0775; C12M 1/00

(52) U.S. Cl. CPC C12N 5/0663; C12N 2533/32; C12N 2521/00; C12M 47/04

(57) ABSTRACT

A device for isolating, retaining, separating and/or purifying mesenchymal stem cells from a heterogeneous population of cells. The device includes at least one inlet, at least one outlet, a housing, and at least one surface located in the housing, the surface having an affinity for mesenchymal stem cells, wherein the surface is in communication with the inlet and the outlet, and the at least one surface includes at least two portions facing each other, and separated by a distance ranging from 1 mm to 1 cm.

### FIELD OF INVENTION

The present invention pertains to the field of stem cells isolation and purification. In particular, the invention relates to methods and apparatuses for mesenchymal stem cells isolation and purification. Another object relates to mesenchymal stem cells isolation and purification, especially for subsequent therapeutic applications.

### BACKGROUND OF INVENTION

Initially, mesenchymal stem cells isolation and purification proposed in the 1960s comprised together washing, heat treatment, chemical treatment by use of enzymes and centrifugation rounds.

However, the use of enzymes, such as collagenase and trypsin, is not recommended on stem cells as it may significantly modify the cells. Furthermore, clinical grade collagenase is expensive.

Consequently, non-enzymatic methods for isolation of mesenchymal stem cells have been developed.

For instance, Van Dongen et al. discloses a procedure to isolate stromal vascular fractions comprising mesenchymal stem cells from condensed lipoaspirate. The fractionation comprises a centrifugation round and then mechanical dissociation. The mechanical dissociation comprises two syringes and a Luer to Luer connector with three 1.4 mm holes. The condensed lipoaspirate is pushed through the Luer to Luer connector forward and backwards thirty times. Said process enables to obtain the stromal vascular fraction from adipose tissue in a sparing way, which is directly available for therapeutic injection. However, the stromal vascular fraction does not comprise purified mesenchymal stem cells but also the extracellular matrix and the microvasculature. In order to purify the stromal vascular fraction Van Dongen et al. proposed anew the digestion with enzymes (namely, collagenase), a centrifugation round and the plating in a culture ware (Van Dongen et al., Wound Repair and Regeneration, Vol. 24, pages 994-1003, 2016).

Baptista et al. concerns the isolation of mesenchymal stem cells by use of two centrifugation steps and subsequent plating in culture dishes for up to 2 weeks. The mesenchymal stem cells are isolated by washing based on their adherence properties on the plastic culture dishes after 2 weeks (Baptista et al., Cryotherapy, Vol. 11, No 6, pages 706-715, 2009). Adhesion-based collection and separation of cells as described by Baptista et al requires time (up to 2 weeks) during which stem cells may start to differentiate.

Documents WO 2004/029221, WO 2015/057159, and WO 2014/000029 disclose microfluidic devices for separating cells. However, micro-channels can create a laminar flow. This would keep cells in suspension flowing in a stable direction. This stability can hinder the chances for cells to interact with the channel surface, therefore the separation of said cells from the sample by adherence of said cells on the channel surface.

Documents US 2010/291534 and WO 2015/057159 disclose filtration devices for separating cells. However, these devices can damage the cells because of the mechanical filtration.

Documents WO 2014/000029 discloses an enzyme method for separating stem cells from a sample. However, this can cause a chemical modification or pollution of the stem cells.

Having regard to the state of the art identified above, one of the object underlying the present invention is to provide a device able to implement a non-enzymatic mechanical method for isolating and purifying mesenchymal stem cells. Another object of the present invention is to provide a simple and fast method therefore.

Several designs have been considered, and a device having several parallel portions of a surface facing each other to create a channel, said surface having an affinity for mesenchymal stem cells, was selected. The device is able to be coupled with pumping means to pump the sample comprising mesenchymal stem cells through the device with an appropriate flow rate. Indeed, using only the actual gravity of the sample, i.e. using for example a vertical spiral, is not sufficient and do not allow the separation of mesenchymal stem cells from the sample. In addition gravity will result in significant differences in flow speed of the sample into the device and inconsistent segregation of the mesenchymal stem cells between two different samples. Furthermore gravity may allow cellular and extra cellular matrix tissue debris to attach to the mesenchymal stem cells and the device's surface.

The present invention also provides a method for isolating and purifying mesenchymal stem cells based on the partitioning of stem cells from other components due to their flow rate on a chosen surface.

In view of the fastness of the method of the invention, the purified stem cells may advantageously be isolated from one patient and reintroduced to said patient during the same surgical procedure.

### SUMMARY

The invention relates to a device for purifying mesenchymal stem cells comprising:
at least one inlet;
at least one outlet;
a housing;
at least one surface located in the housing, said surface having an affinity for mesenchymal stem cells;
   wherein said surface is in communication with the inlet and the outlet; and
   wherein the at least one surface comprises at least two portions facing each other, and separated by a distance ranging from 1 mm to 1 cm.

In one embodiment, the at least one surface comprises a material selected in the group of polymeric material such as polystyrene, (PS), polyamine, polycarbonate (PC), poly-D-lysine (PDL), polycaprolactone, or inorganic material such as glass.

In one embodiment, the at least one surface is smooth.

In one embodiment, the at least two portions are slides or coverslips.

In one embodiment, the at least two portions are facing each other in staggered rows.

In one embodiment, the at least two portions are parallel to each other, or describe an angle to each other ranging from 0° to 30°.

In one embodiment, the at least one surface is a planar spiral.

The invention relates to a method for isolating and purifying mesenchymal stem cells from a sample comprising mesenchymal stem cells, comprising the following steps:
pushing said sample in the device of the invention, at a first flow rate allowing to separate said sample into a first remaining sample comprising mesenchymal stem cells on the at least one surface and into a second resulting solution being evacuated from the device;
evacuating the second resulting solution from the device;
pumping a saline solution in the device at a second flow rate different from the first flow rate; and
collecting the saline solution comprising isolated and purified mesenchymal stem cells;
   wherein the first flow rate is slower than the second flow rate;
   wherein the ratio between the first flow rate and the second flow rate is ranging from 2 to 50.

In one embodiment, the method further comprises a centrifugation step before pumping the sample in the device.

In one embodiment, the first flow rate is ranging from 10 to 150 ml/min.

In one embodiment, the second flow rate is ranging from 100 to 500 ml/min.

The present invention also relates to the use of the device of the invention for purifying adipose-derived mesenchymal stem cells, bone marrow-derived stem cells, blood-derived mesenchymal stem cells, blood umbilical cord stem cells, molar stem cells, amniotic fluid stem cells, follicular stem cells, or human embryonic stem cells (hESC) obtained without the destruction of an embryo.

The present invention also relates to a system for isolation and purification of mesenchymal stem cells, comprising:
a device of the invention;
a first supplying reservoir fluidly connected to at least one inlet of the device and configured to comprise at least one sample comprising mesenchymal stem cells;
a second supplying reservoir fluidly connected to at least one inlet of the device and configured to comprise at least one saline solution;
at least one collecting reservoir fluidly connected to at least one outlet of the device;
at least one pumping means fluidly connected to at least one supplying reservoir; and
at least one flow controller configured to control the flow rate of a fluid pumped through the inlet of the device.

In one embodiment, the system further comprises a flowcytometer.

In one embodiment, the system further comprises a temperature controller.

The present invention relates to a device for isolating, retaining, separating and/or purifying mesenchymal stem cells from a heterogeneous population of cells comprising:
at least one inlet;
at least one outlet;
a housing;
at least one surface located in the housing, said surface having an affinity for mesenchymal stem cells;
   wherein said surface is in communication with the inlet and the outlet; and
   wherein the at least one surface comprises at least two portions facing each other, and separated by a distance ranging from 1 mm to 1 cm.

The present invention relates to a method for isolating, retaining, separating and/or purifying mesenchymal stem cells from a heterogeneous population of cells comprising mesenchymal stem cells, comprising the following steps:
pushing said heterogeneous population of cells in a device of the invention, at a first flow rate allowing to separate said heterogeneous population of cells into a first remaining sample comprising mesenchymal stem cells on the at least one surface and into a second resulting solution being evacuated from the device;
evacuating the second resulting solution from the device;
pumping a saline solution in the device at a second flow rate different from the first flow rate; and
collecting the saline solution comprising isolated, retained, separated and/or purified mesenchymal stem cells;
   wherein the first flow rate is slower than the second flow rate;
   wherein the ratio between the first flow rate and the second flow rate is ranging from 2 to 50.

The present invention relates to a use of the device of the invention for isolating, retaining, separating, and/or purifying adipose-derived mesenchymal stem cells, bone marrow-derived mesenchymal stem cells, blood-derived mesenchymal stem cells, blood umbilical cord stem cells, molar stem cells, amniotic fluid stem cells, follicular stem cells, or human embryonic stem cells (hESC) obtained without the destruction of an embryo.

The present invention relates to a system for isolation, retention, separation and/or purification of mesenchymal stem cells from at least one heterogeneous population of cells, comprising:
a device of the invention;
a first supplying reservoir fluidly connected to at least one inlet of the device and configured to comprise at least one heterogeneous population of cells sample comprising mesenchymal stem cells;
a second supplying reservoir fluidly connected to at least one inlet of the device and configured to comprise at least one saline solution;
at least one collecting reservoir fluidly connected to at least one outlet of the device;
at least one pumping means fluidly connected to at least one supplying reservoir; and
   at least one flow controller configured to control the flow rate of a fluid pumped through the inlet of the device.

### Definitions

In the present invention, the following terms have the following meanings:
“Automated”, “Semi-automated” and “Manual” refer to operating methods. “Automated” refers to an operating method, wherein all the steps are independent of external control. “Semi-automated” refers to an operating method, wherein at least one step is automated and at least one step is manual. “Manual” refers to an operating method, wherein all the steps are operated by hand.
“Isolation” refers to the extraction of cells from their original environment. A suspension of stem cells is referred to as isolated if it is free of pre-adipocytes, endothelial cells, smooth muscle cells, pericytes, fibroblasts, erythrocytes, B and T cells, macrophages, monocytes or mast cells.
“Mesenchymal stem cells” refers to multipotent stromal cells that can differentiate into a variety of cell types. In mammals, they are found in multicellular bone marrow, adipose tissue (referred to as adipose-derived mesenchymal stem cells), blood umbilical cord, molar cells, amniotic fluid, follicular cells. “Mesenchymal stem cells” may refer to bone marrow stem cells, adipose-derived mesenchymal stem cells, blood umbilical cord stem cells, molar stem cells, amniotic fluid stem cells, follicular stem cells, or human embryonic stem cells (hESC) obtained without the destruction of an embryo using a method such as for example the one described in Chung et al., Cell Stem Cell, Vol. 2 (2), pages 113-117, 2008.
“Microfluidic” refers to a structure comprising at least one channel having at least one dimension ranging from 1 to 100 nm.
“Purification” refers herein to the preparation of a pure population of one type of cells. A suspension of stem cells is referred to as purified if it is free of cellular debris or extracellular matrix product.
“Saline solution” refers to a mixture of sodium chloride in water.
“Sample” refers to the cells suspension destined to be purified by the device or system of the invention.

### DETAILED DESCRIPTION

The following detailed description will be better understood when read in conjunction with the drawings. For the purpose of illustrating, the apparatus is shown in the preferred embodiments. It should be understood, however that the application is not limited to the precise arrangements, structures, features, embodiments, and aspect shown. The drawings are not drawn to scale and are not intended to limit the scope of the claims to the embodiments depicted. Accordingly, it should be understood that where features mentioned in the appended claims are followed by reference signs, such signs are included solely for the purpose of enhancing the intelligibility of the claims and are in no way limiting on the scope of the claims.

In a first aspect illustrated in FIGS. 1-5, the present invention relates to a device 1 for isolating, retaining, separating and/or purifying mesenchymal stem cells from a heterogeneous population of cells.

Said device 1 comprises:
at least one inlet 11;
at least one outlet 12;
a housing 13;
at least one surface 14 located in the housing 13, said surface 14 having an affinity for mesenchymal stem cells;

The surface 14 is in communication with the inlet 11 and the outlet 12, and comprises at least two portions 141, 142 facing each other, and separated by a distance ranging from 1 mm to 1 cm, from 2 mm to 1 cm, from 3 mm to 1 cm, from 4 mm to 1 cm, from 5 mm to 1 cm, from 6 mm to 1 cm, from 6 mm to 1 cm, or from 8 mm to 1 cm. The surface 14 is in communication with the inlet 11 and the outlet 12, and comprises at least two portions 141, 142 facing each other, and separated by a distance ranging from 1 mm to 8 mm, from 1 mm to 7 mm, from 1 mm to 6 mm, from 1 mm to 5 mm, from 1 mm to 4 mm, from 1 mm to 3 mm, or from 1 mm to 4 mm.

The distance between the at least two portions 141, 142 allows the creation of a flow with a certain rate when a fluid is introduced through the inlet 11 of the device 1. This flow rate can be increased by the use of a pumping means to pull the fluid through the device 1, i.e. from the inlet 11 to the outlet 12.

When a sample comprising mesenchymal stem cells is pushed through the device 1, using for example pumping means, the mesenchymal stem cells decelerate as the surface 14 has an affinity for them, while other cells and particles in the sample are carried away allowing to separate the sample into a first remaining sample comprising mesenchymal stem cells located near the surface 14 and into a second resulting solution that can be evacuated from the device 1. Said second resulting solution comprises particles, cellular debris (etc. . . . ) that were not attracted by the surface 14. The first remaining sample is collected by pushing a saline solution through the device 1.

The isolation, retaining, separation and/or purification of mesenchymal stem cells in the device 1 is based on the adherence properties of mesenchymal stem cells on polymer materials.

The sample of mesenchymal stem cells has to be pumped in the device 1 to ensure a sufficient flow rate. Using only the actual gravity of the sample is not sufficient and do not allow a fine control on the flow rate of said sample in the device 1. In addition gravity will result in significant differences in flow speed of the sample into the device and inconsistent segregation of the mesenchymal stem cells between two different samples. Furthermore gravity may allow cellular and/or extra cellular matrix tissue debris to attach to the mesenchymal stem cells and the device's surface.

The first flow rate is configured to be fast enough so that the mesenchymal stem cells do not adhere to the surface, but slow enough so that said mesenchymal stem cells decelerate in the vicinity of the surface. The advantage is that the mesenchymal stem cells are retained for a short time on the surface, this time being short enough to prevent the differentiation of the stem cells, while the other cells or cellular debris are evacuated from the device 1. Thus, the differentiated flow of the mesenchymal stem cells compared to other cells or cellular debris comprised in the sample allows for the isolation and purification of said mesenchymal stem cells.

According to the Applicant, by advantageously selecting the first flow rate, the material of the surface, the mesenchymal stem cells do not adhere on the surface but only slow down due to their natural attraction to the surface. Thus, stem cells are retained due to their attraction to the surface while other components of the first flow (cellular debris, extracellular matrix product, pre-adipocytes . . . ) are drained away at a rate equal to the first flow rate. However, it is important that the mesenchymal stem cells do not adhere on the surface so that they are not attached to it; this prevents the risk of differentiation of the stem cells in the device 1. The length of the surface, i.e. the length of the route that the fluid has to take, the surface composition defining the affinity of the cells for said surface, the temperature of the surface and/or the cell suspension, and the dilution levels of the saline solution, may also be of importance as the longer the surface is, the more the stem cells may be retained.

The second flow rate is configured to be faster than the first flow rate, and fast enough so that the mesenchymal stem cells are carried away with said second flow.

The purified mesenchymal stem cells may be delivered immediately to different specialists for the treatment of a variety of conditions, for instance in orthopedics (joint diseases, bone grafts), ophthalmology (macular degeneration, glaucoma), uro-gynecology (urinary incontinence, erectile dysfunction), plastic surgery (face peels, hair regrowth, scars, lipofilling, lipostructure, breast augmentation), wound healing, salivary glands stimulation after radiotherapy, peripheral arterial disease. The purified mesenchymal stem cells may be reinjected in the subject who provided the sample of adipose tissue. They may be injected in joints, salivary glands, retina, skin, muscle, or mixed with bone graft. These cells may be used for any tissue regeneration procedure or method. They may also be topically applied on wounds, burn wounds, ulcers, after chemical or mechanical face peeling. They may also be used for in vitro tissue regeneration.

The saline solution comprising isolated, retained, separated and/or purified mesenchymal stem cells is also referred hereafter as a suspension of isolated, retained, separated and/or purified mesenchymal stem cells.

In one embodiment, the flow created when the suspension of cells and when the saline solution are each pushed through the device 1 is not a laminar flow.

In one embodiment, the flow created when the suspension of cells and when the saline solution are each pushed through the device 1 is a turbulent flow. This embodiment is particularly advantageous as a turbulent flow allowing the reduction of speed of cells of interest and the resulting separation of said cells from the sample. A turbulent flow also reduces the time needed to separate cells of interest from a sample as it increases the chance of said cells to be close to the surface 14.

In one embodiment, the flow in the device 1 is a turbulent flow.

In one embodiment, the flow in the device 1 is a dynamic flow.

In one embodiment, the flow in the device 1 is a continuous flow.

In one embodiment, the device 1 is configured to receive a turbulent, dynamic and/or continuous flow of a population of cells.

The device 1 is not a microfluidic structure, i.e. does not comprise a channel having a width or diameter less than 1 mm Indeed, micro-channels increase viscosity of fluids and can create a laminar flow. This would keep cells in suspension flowing in a stable direction. This stability can hinder the chances for cells to interact with the channel surface, therefore preventing the reduction of speed of said cells and the resulting separation of said cells from the sample. The process of separation of cells from a sample would then take more time with a laminar flow and/or a microfluidic structure.

A turbulent flow, needed and essential for separating cells of interest from a sample, cannot be achieved in a microfluidic structure. Indeed, using bigger channels than micro-channels in device 1 decreases viscosity and allows for turbulent flow. This physical property is antithetical to microfluidics where laminar flow is induced by channel size. In the device 1, cells would have an increased chance of interacting with the surface geography, therefore allowing the reduction of speed of said cells and the resulting separation of said cells from the sample.

In one embodiment, the device 1 does not comprise a microfluidic structure.

In one embodiment, the device 1 is sterile.

In one embodiment, the device 1 is disposable.

In one embodiment, the device 1 is reusable. In this embodiment, the device 1 can be sterilized after use.

In one embodiment, the mesenchymal stem cells are adipose-derived mesenchymal stem cells, bone marrow-derived mesenchymal stem cells, blood-derived mesenchymal stem cells, blood umbilical cord stem cells, molar stem cells, amniotic fluid stem cells, follicular stem cells, or human embryonic stem cells (hESC) obtained without the destruction of an embryo using a method such as for example the one described in Chung et al., Cell Stem Cell, Vol. 2 (2), pages 113-117, 2008.

In one embodiment, the at least one inlet 11 and the at least one outlet 12 have a circular or ovoidal shape.

In one embodiment, the at least one inlet 11 and the at least one outlet 12 have the same shape.

In one embodiment, the at least one inlet 11 and the at least one outlet 12 have two distinct shapes.

In one embodiment, the at least one inlet 11 and/or the at least one outlet 12 have at least one dimension ranging from 1 mm to 5 cm. In this embodiment, the at least one dimension may be the length.

In one embodiment, the at least one inlet 11 and/or the at least one outlet 12 have at least one dimension of at least 1 mm, 1.1 mm, 1.2 mm, 1.3 mm, 1.4 mm, 1.5 mm, 1.6 mm, 1.7 mm, 1.8 mm, 1.9 mm, 2 mm, 2.1 mm, 2.2 mm, 2.3 mm, 2.4 mm, 2.5 mm, 2.6 mm, 2.7 mm, 2.8 mm, 2.9 mm, 3 mm, 3.1 mm, 3.2 mm, 3.3 mm, 3.4 mm, 3.5 mm, 3.6 mm, 3.7 mm, 3.8 mm, 3.9 mm, 4 mm, 4.1 mm, 4.2 mm, 4.3 mm, 4.4 mm, 4.5 mm, 4.6 mm, 4.7 mm, 4.8 mm, 4.9 mm, 5 mm, 5.1 mm, 5.2 mm, 5.3 mm, 5.4 mm, 5.5 mm, 5.6 mm, 5.7 mm, 5.8 mm, 5.9 mm, 6 mm, 6.1 mm, 6.2 mm, 6.3 mm, 6.4 mm, 6.5 mm, 6.6 mm, 6.7 mm, 6.8 mm, 6.9 mm, 7 mm, 7.1 mm, 7.2 mm, 7.3 mm, 7.4 mm, 7.5 mm, 7.6 mm, 7.7 mm, 7.8 mm, 7.9 mm, 8 mm, 8.1 mm, 8.2 mm, 8.3 mm, 8.4 mm, 8.5 mm, 8.6 mm, 8.7 mm, 8.8 mm, 8.9 mm, 9 mm, 9.1 mm, 9.2 mm, 9.3 mm, 9.4 mm, 9.5 mm, 9.6 mm, 9.7 mm, 9.8 mm, 9.9 mm, 1 cm, 1.1 cm, 1.2 cm, 1.3 cm, 1.4 cm, 1.5 cm, 1.6 cm, 1.7 cm, 1.8 cm, 1.9 cm, 2 cm, 2.1 cm, 2.2 cm, 2.3 cm, 2.4 cm, 2.5 cm, 2.6 cm, 2.7 cm, 2.8 cm, 2.9 cm, 3 cm, 3.1 cm, 3.2 cm, 3.3 cm, 3.4 cm, 3.5 cm, 3.6 cm, 3.7 cm, 3.8 cm, 3.9 cm, 4 cm, 4.1 cm, 4.2 cm, 4.3 cm, 4.4 cm, 4.5 cm, 4.6 cm, 4.7 cm, 4.8 cm, 4.9 cm, or 5 cm.

In one embodiment, the at least one inlet 11 and/or the at least one outlet 12 have a radius ranging from 1 to 5 mm, from 2 to 5 mm, from 3 to 5 mm, or from 4 to 5 mm. In one embodiment, the at least one inlet 11 and/or the at least one outlet 12 have a radius ranging from 1 to 4 mm, from 1 to 3 mm, or from 1 to 2 mm.

In one embodiment, the at least one inlet 11 and the at least one outlet 12 have the same radius.

In one embodiment, the at least one inlet 11 and the at least one outlet 12 have two distinct radii.

In one embodiment, the at least one inlet 11 and the at least one outlet 12 are located at the same level on the device 1.

In one embodiment, the at least one inlet 11 and the at least one outlet 12 are located at a distinct level on the device 1, said at least one outlet 12 being located at a level inferior to the one of the at least one inlet 11.

According to one embodiment, the inlet 11 and the outlet 12 are combined. In this embodiment, the inlet 11 can also be an outlet.

According to one embodiment, the device 1 comprises two inlets 11. According to another embodiment, the device 1 comprises two outlets 12.

In one embodiment, the device 1 further comprises a luer-lock connection at the at least one inlet 11 and/or at the at least one outlet 12.

In one embodiment, the device 1 can be connected to a syringe at the inlet 11, said syringe being configured to push a fluid in said device 1.

In one embodiment, the at least one surface 14 comprises a material selected in the group of polymeric material such as polystyrene (PS), polyamine, polycarbonate (PC), poly-D-lysine (PDL), polycaprolactone, or inorganic material such as glass.

In one embodiment, the material of the surface 14 is polystyrene. The mesenchymal stem cells are known to have adherent properties on polystyrene.

In one embodiment, the material of the surface 14 is 100% polystyrene, i.e. pure virgin polystyrene.

In one embodiment, the structure of the surface 14 is modified using UV treatment, ultrasounds, plasma treatment, high voltage corona discharge, gas-plasma, high energy microwave plasma or any other plasma surface activation methods. These modifications have the potential to increase the adhesive properties of the mesenchymal stem cells on said surface 14.

In one embodiment, the structure of the surface 14 is modified and/or functionalized by pushing through the device 1 a serous product prior pushing through the device the sample comprising a heterogeneous population of cells. In this embodiment, the serous product can be a serous product originating from the subject. In this embodiment, the serous product comprises preferably a blood product, preferably blood plasma. This embodiment is particularly advantageous as pushing through a serous product from the subject itself in the device 1 allows the serous product of the subject to adhere to the surface 14, increasing the affinity of said surface for the mesenchymal stem cells of the subject.

In one embodiment, the surface 14 is not functionalized. In this embodiment, the surface 14 is not subjected to any surface treatment, and is not coated with a material that could detach and pollute the mesenchymal stem cells.

In one embodiment, the surface 14 is functionalized by coating said surface with organic material such as for example collagen; laminin; fribronectin; mucopolysaccharides such as for example heparin sulfate, hyaluronidate, chondroitin sulfate, or a mixture thereof; or synthetic polymers, such as for example poly-D-lysine (PDL); or a mixture thereof.

In one embodiment, the surface 14 is sterile.

In one embodiment, the surface 14 is specific for mesenchymal stem cells.

In one embodiment, the surface 14 is adapted to mesenchymal stem cells adherence.

In one embodiment, the surface 14 is made by 3D-printing.

In one embodiment, the surface 14 is smooth. In this embodiment, the surface 14 does not comprise any protrusion or depression.

In one embodiment, the surface 14 is rough.

In one embodiment, the surface 14 comprises at least one protrusion and/or at least one depression.

The roughness of the surface is measured using a mechanical profilometer (diamond stylus) or an optical profilometer (white light interferometer or laser scanning confocal microscope) making a surface profile measurement, optical microscopy or any other method known in the art.

The roughness refers to surface defects such as irregularities on the surface defined by comparison with a medium line and classified in two categories: profile asperities or profile peaks, and profile valleys. A profile peak refers to an outwardly directed (from material to surrounding medium) portion of the assessed surface profile connecting two adjacent points of the intersection of said profile with the medium line. A profile valley refers to an inwardly directed (from surrounding medium to material) portion of the assessed surface profile connecting two adjacent points of the intersection of said profile with the medium line.

In one embodiment, the surface 14 has an arithmetical mean deviation Rₐ ranging from 100 to 10000 nm, from 100 to 5000 nm, from 100 to 1000 nm, from 500 to 10000 nm, from 1000 to 10000 nm, or from 5000 to 10000 nm.

In one embodiment, the surface 14 has a root mean square deviation R_{q} ranging from 100 to 10000 nm, from 100 to 5000 nm, from 100 to 1000 nm, from 500 to 10000 nm, from 1000 to 10000 nm, or from 5000 to 10000 nm.

In one embodiment, the surface 14 has a maximum profile peak height (Rₚ) ranging from 100 to 10000 nm, from 100 to 5000 nm, from 100 to 1000 nm, from 500 to 10000 nm, from 1000 to 10000 nm, or from 5000 to 10000 nm.

In one embodiment, the surface 14 has a maximum profile valley depth (Rᵥ) ranging from 100 to 10000 nm, from 100 to 5000 nm, from 100 to 1000 nm, from 500 to 10000 nm, from 1000 to 10000 nm, or from 5000 to 10000 nm.

In one embodiment, the surface 14 has a mean profile element height (Rₑ), i.e. sum of the height of the profile peak and the depth of the profile valley, ranging from 100 to 10000 nm, from 100 to 5000 nm, from 100 to 1000 nm, from 500 to 10000 nm, from 1000 to 10000 nm, or from 5000 to 10000 nm.

In one embodiment, the surface 14 has a surface roughness due to an arrangement of nanofibers.

In one embodiment the nanofibers can be randomly organized or oriented.

In one embodiment the nanofibers have a diameter ranging from 100 to 10000 nm, from 100 to 5000 nm, from 100 to 1000 nm, from 500 to 10000 nm, from 1000 to 10000 nm, or from 5000 to 10000 nm.

In the embodiment shown on FIG. 5, the at least one surface 14 is a planar spiral.

In one embodiment, the at least one surface 14 is not a vertical spiral, does not comprise any vertical helicoidal portion.

In one embodiment, the at least two portions 141, 142 each have a length ranging from 5 cm to 15 cm, from 5 cm to 10 cm, from 5 cm to 9 cm, from 5 cm to 8 cm, or from 5 cm to 7 cm.

In one embodiment, the at least two portions 141, 142 each have a length of at least 5 cm, 6 cm, 7 cm, 8 cm, 9 cm, 10 cm, 11 cm, 12 cm, 13 cm, 14 cm, 15 cm, 16 cm, 17 cm, 18 cm, 19 cm, 20 cm, 21 cm, 22 cm, 23 cm, 24 cm, 25 cm, 26 cm, 27 cm, 28 cm, 29 cm, 30 cm, 31 cm, 32 cm, 33 cm, 34 cm, 35 cm, 36 cm, 37 cm, 38 cm, 39 cm, 40 cm, 41 cm, 42 cm, 43 cm, 44 cm, 45 cm, 46 cm, 47 cm, 48 cm, 49 cm, 50 cm, 51 cm, 52 cm, 53 cm, 54 cm, 55 cm, 56 cm, 57 cm, 58 cm, 59 cm, 60 cm, 61 cm, 62 cm, 63 cm, 64 cm, 65 cm, 66 cm, 67 cm, 68 cm, 69 cm, 70 cm, 71 cm, 72 cm, 73 cm, 74 cm, 75 cm, 76 cm, 77 cm, 78 cm, 79 cm, 80 cm, 81 cm, 82 cm, 83 cm, 84 cm, 85 cm, 86 cm, 87 cm, 88 cm, 89 cm, 90 cm, 91 cm, 92 cm, 93 cm, 94 cm, 95 cm, 96 cm, 97 cm, 98 cm, 99 cm, or 100 cm.

In one embodiment, the at least two portions 141, 142 each have a width ranging from from 5 cm to 15 cm, from 5 cm to 10 cm, from 5 cm to 9 cm, from 5 cm to 8 cm, or from 5 cm to 7 cm.

In one embodiment, the at least two portions 141, 142 each have a width of at least 5 cm, 6 cm, 7 cm, 8 cm, 9 cm, 10 cm, 11 cm, 12 cm, 13 cm, 14 cm, 15 cm, 16 cm, 17 cm, 18 cm, 19 cm, 20 cm, 21 cm, 22 cm, 23 cm, 24 cm, 25 cm, 26 cm, 27 cm, 28 cm, 29 cm, 30 cm, 31 cm, 32 cm, 33 cm, 34 cm, 35 cm, 36 cm, 37 cm, 38 cm, 39 cm, 40 cm, 41 cm, 42 cm, 43 cm, 44 cm, 45 cm, 46 cm, 47 cm, 48 cm, 49 cm, 50 cm, 51 cm, 52 cm, 53 cm, 54 cm, 55 cm, 56 cm, 57 cm, 58 cm, 59 cm, 60 cm, 61 cm, 62 cm, 63 cm, 64 cm, 65 cm, 66 cm, 67 cm, 68 cm, 69 cm, 70 cm, 71 cm, 72 cm, 73 cm, 74 cm, 75 cm, 76 cm, 77 cm, 78 cm, 79 cm, 80 cm, 81 cm, 82 cm, 83 cm, 84 cm, 85 cm, 86 cm, 87 cm, 88 cm, 89 cm, 90 cm, 91 cm, 92 cm, 93 cm, 94 cm, 95 cm, 96 cm, 97 cm, 98 cm, 99 cm, or 100 cm.

In one embodiment, the at least two portions 141, 142 each have a thickness ranging from 1 mm to 5 mm, from 1 mm to 4 mm, from 1 mm to 3 mm, or from 1 mm to 2 mm.

In one embodiment, illustrated in FIGS. 1-4, the at least two portions 141, 142 are substantially parallel to each other.

In one embodiment, the at least two portions 141, 142 are parallel to each other.

In one embodiment, the at least two portions 141, 142 describe an angle to each other ranging from 0° to 30°, from 0° to 25°, from 0° to 20°, from 0° to 15°, from 0° to 10°, or from 0° to 5°. The angle allows the suspension to flow from one portion to the other.

In one embodiment illustrated in FIGS. 1-2, the at least two portions 141, 142 are slides or coverslips.

In one embodiment illustrated in FIG. 1 and FIG. 3, the at least two portions 141, 142 are facing each other in staggered rows.

In the embodiment shown on FIG. 1, the device 1 comprises:
a housing 13 in a shape of a rectangular parallelepiped;
an inlet 11 and an outlet 12 located on opposite sides and opposite height of the housing 13; and
parallel slides arranged in staggered rows; the surface of said slides having an affinity for mesenchymal stem cells.

In the embodiment shown on FIG. 2, the device 1 comprises:
a housing 13 in a shape of a rectangular parallelepiped;
an inlet 11 and an outlet 12 located on opposite sides and at the same height of the housing 13; and
parallel slides; the surface of said slides having an affinity for mesenchymal stem cells.

In the embodiment shown on FIG. 12, the device 1 comprises:
a housing 13 in a shape of a rectangular parallelepiped;
an inlet 11 and an outlet 12 located on opposite sides and at the same height of the housing 13; and
at least two internal surfaces facing each other and having an affinity for mesenchymal stem cells.

A sample comprising mesenchymal stem cells is pushed through in the device 1 through the inlet 11, using for example pumping means. The sample runs from slide to slide. The mesenchymal stem cells decelerate and are retained in the vicinity of the surface of the slides, while other components (cellular debris, particles . . . ) continue their path along the slides until their evacuation through the outlet 12. The purified mesenchymal stem cells can then be evacuated from the device 1 and collected by pumping a saline solution through the inlet 11. Indeed, the flow of saline solution will carry the mesenchymal stem cells away from the surface of the slides.

In the embodiment shown on FIG. 3, the device 1 comprises:
a housing 13 in a shape of a tube with a conic or a rounded end;
an inlet 11 and an outlet 12 located at the same level of the device 1; and
parallel slides arranged in staggered rows; the surface of said slides having an affinity for mesenchymal stem cells;
   wherein the housing 13 is divided in two on its length, so that when a sample is pumped in the device 1 through the inlet 11, it goes in the front part of the device 1 and runs from slide to slide. The mesenchymal stem cells decelerate and are retained in the vicinity of the surface of the slides, while other components (cellular debris, particles . . . ) continue their path along the slides until their passage to the back part of the device 1 and finally their evacuation through the outlet 12. The purified mesenchymal stem cells can then be evacuated from the device 1 and collected by pumping a saline solution through the inlet 11. Indeed, the flow of saline solution will carry the mesenchymal stem cells away from the surface of the slides.

In the embodiment shown on FIG. 4, the device 1 comprises:
a housing 13 in a shape of a rectangular parallelepiped;
an inlet 11 and an outlet 12 located on the same side and height of the housing 13; and
parallel portions 141, 142 of surface arranged to create a channel leading from the inlet 11 to the outlet 12; the surface having an affinity for mesenchymal stem cells.

The sample has to run through the channel to go from the inlet 11 to the outlet 12. The mesenchymal stem cells decelerate and are retained in the vicinity of the surface, while other components (cellular debris, particles . . . ) continue their path along the slides until their evacuation through the outlet 12. The purified mesenchymal stem cells can then be evacuated from the device 1 and collected by pumping a saline solution through the inlet 11. Indeed, the flow of saline solution will carry the mesenchymal stem cells away from the surface of the slides. The length of the channel is judiciously chosen to allow an optimal hold of the mesenchymal stem cells in the device 1 while other components of the sample are fully evacuated.

In one embodiment, the device 1 comprises a cylindrical tube configured to be centrifuged. This embodiment is particularly advantageous as it increases closeness and/or contact between mesenchymal stem cells and the surface 14.

In one embodiment illustrated in FIGS. 10 A-B and 11 A-D, the device 1 comprises a housing 13 being a cylindrical tube configured to be rotated. In this embodiment, the device 1 may comprise a rotating mechanism 15. This embodiment is particularly advantageous as it increases closeness and/or contact between mesenchymal stem cells and the surface 14. This is illustrated in FIGS. 11 B and D: upon rotation, the flow 3 of cell suspension is pushed towards the surface 14, cells 31 are then brought closer to the surface 14.

In one embodiment, the surface 14 comprises 2, 3, 4, 5, 6, 7, 8, 9, or 10 portions facing each other and separated by the distance described hereabove.

In one embodiment, the at least two portions 141, 142 forms a channel where a fluid can flow between said at least two portions 141, 142.

In one embodiment, the channel is not a microfluidic channel.

In one embodiment, the channel has a portion having at least one dimension ranging from 1 mm to 1 cm.

In one embodiment, the channel has a portion having at least one dimension of at least 1 mm, 1.1 mm, 1.2 mm, 1.3 mm, 1.4 mm, 1.5 mm, 1.6 mm, 1.7 mm, 1.8 mm, 1.9 mm, 2 mm, 2.1 mm, 2.2 mm, 2.3 mm, 2.4 mm, 2.5 mm, 2.6 mm, 2.7 mm, 2.8 mm, 2.9 mm, 3 mm, 3.1 mm, 3.2 mm, 3.3 mm, 3.4 mm, 3.5 mm, 3.6 mm, 3.7 mm, 3.8 mm, 3.9 mm, 4 mm, 4.1 mm, 4.2 mm, 4.3 mm, 4.4 mm, 4.5 mm, 4.6 mm, 4.7 mm, 4.8 mm, 4.9 mm, 5 mm, 5.1 mm, 5.2 mm, 5.3 mm, 5.4 mm, 5.5 mm, 5.6 mm, 5.7 mm, 5.8 mm, 5.9 mm, 6 mm, 6.1 mm, 6.2 mm, 6.3 mm, 6.4 mm, 6.5 mm, 6.6 mm, 6.7 mm, 6.8 mm, 6.9 mm, 7 mm, 7.1 mm, 7.2 mm, 7.3 mm, 7.4 mm, 7.5 mm, 7.6 mm, 7.7 mm, 7.8 mm, 7.9 mm, 8 mm, 8.1 mm, 8.2 mm, 8.3 mm, 8.4 mm, 8.5 mm, 8.6 mm, 8.7 mm, 8.8 mm, 8.9 mm, 9 mm, 9.1 mm, 9.2 mm, 9.3 mm, 9.4 mm, 9.5 mm, 9.6 mm, 9.7 mm, 9.8 mm, 9.9 mm, or 1 cm.

The at least one dimension may be the length or the width of the channel.

In one embodiment, the channel has a length ranging from 10 cm to 100 cm.

In one embodiment, the channel has a length of at least 10 cm, 11 cm, 12 cm, 13 cm, 14 cm, 15 cm, 16 cm, 17 cm, 18 cm, 19 cm, 20 cm, 21 cm, 22 cm, 23 cm, 24 cm, 25 cm, 26 cm, 27 cm, 28 cm, 29 cm, 30 cm, 31 cm, 32 cm, 33 cm, 34 cm, 35 cm, 36 cm, 37 cm, 38 cm, 39 cm, 40 cm, 41 cm, 42 cm, 43 cm, 44 cm, 45 cm, 46 cm, 47 cm, 48 cm, 49 cm, 50 cm, 51 cm, 52 cm, 53 cm, 54 cm, 55 cm, 56 cm, 57 cm, 58 cm, 59 cm, 60 cm, 61 cm, 62 cm, 63 cm, 64 cm, 65 cm, 66 cm, 67 cm, 68 cm, 69 cm, 70 cm, 71 cm, 72 cm, 73 cm, 74 cm, 75 cm, 76 cm, 77 cm, 78 cm, 79 cm, 80 cm, 81 cm, 82 cm, 83 cm, 84 cm, 85 cm, 86 cm, 87 cm, 88 cm, 89 cm, 90 cm, 91 cm, 92 cm, 93 cm, 94 cm, 95 cm, 96 cm, 97 cm, 98 cm, 99 cm, or 100 cm.

In one embodiment, the channel has a width ranging from 1 mm to 10 mm, from 1 mm to 9 mm, from 1 mm to 8 mm, from 1 mm to 7 mm, from 1 mm to 6 mm, or from 1 mm to 5 mm.

In another aspect, the present invention relates to a system for isolation, retention, separation and/or purification of mesenchymal stem cells from at least one heterogeneous population of cells.

As illustrated on FIG. 7, said system 2 comprises:
a device 1 as described hereabove;
a first supplying reservoir 21 fluidly connected to at least one inlet 11 of the device 1 and configured to comprise at least one sample comprising mesenchymal stem cells;
a second supplying reservoir 22 fluidly connected to at least one inlet 11 of the device 1 and configured to comprise at least one saline solution;
at least one collecting reservoir 23 fluidly connected to at least one outlet 12 of the device 1;
at least one pumping means 24 fluidly connected to at least one supplying reservoir 21, 22; and
at least one flow controller 25 configured to control the flow rate of a fluid pumped through the inlet 11 of the device 1.

As illustrated on FIG. 7, said system 2 comprises:
a device 1 as described hereabove;
a first supplying reservoir 21 fluidly connected to at least one inlet 11 of the device 1 and configured to comprise at least one heterogeneous population of cells comprising mesenchymal stem cells;
a second supplying reservoir 22 fluidly connected to at least one inlet 11 of the device 1 and configured to comprise at least one saline solution;
at least one collecting reservoir 23 fluidly connected to at least one outlet 12 of the device 1;
at least one pumping means 24 fluidly connected to at least one supplying reservoir 21, 22; and
at least one flow controller 25 configured to control the flow rate of a fluid pumped through the inlet 11 of the device 1.

In one embodiment illustrated on FIG. 9, the system 2 further comprises a flowcytometer 26. The flowcytometer 26 is configured to measure the quantity and/or concentration of mesenchymal stem cells in the suspension of purified mesenchymal stem cells. In this embodiment, the quantity of purified mesenchymal stem cells may be measured for each sample. This will allow a personalized and more efficient use of the resulting suspension of purified mesenchymal stem cells. Indeed, this may lead the user to the decision to take an additional sample to have more cells available and reach a therapeutic threshold.

In one embodiment, the system 2 further comprises a temperature controller. In this embodiment, the system 2 works at a controlled temperature. Said temperature ranges from 10° C. to 37° C. In one embodiment, the temperature controller is located in a first supplying reservoir 21 fluidly before the inlet 11.

In one embodiment, the system 2 is portable. In this embodiment, said system 2 may be easily transported.

In one embodiment, the system 2 is compliant with requirements of a sterile environment.

In one embodiment, the first and/or second supplying reservoir 21, 22 is fluidly connected to the at least one inlet 11 of the device 1 by clipping, gluing, at least one tube or any attachment means.

In one embodiment, the at least one tube has a length ranging from 10 cm to 100 cm.

In one embodiment, the at least one tube has a length of at least 10 cm, 11 cm, 12 cm, 13 cm, 14 cm, 15 cm, 16 cm, 17 cm, 18 cm, 19 cm, 20 cm, 21 cm, 22 cm, 23 cm, 24 cm, 25 cm, 26 cm, 27 cm, 28 cm, 29 cm, 30 cm, 31 cm, 32 cm, 33 cm, 34 cm, 35 cm, 36 cm, 37 cm, 38 cm, 39 cm, 40 cm, 41 cm, 42 cm, 43 cm, 44 cm, 45 cm, 46 cm, 47 cm, 48 cm, 49 cm, 50 cm, 51 cm, 52 cm, 53 cm, 54 cm, 55 cm, 56 cm, 57 cm, 58 cm, 59 cm, 60 cm, 61 cm, 62 cm, 63 cm, 64 cm, 65 cm, 66 cm, 67 cm, 68 cm, 69 cm, 70 cm, 71 cm, 72 cm, 73 cm, 74 cm, 75 cm, 76 cm, 77 cm, 78 cm, 79 cm, 80 cm, 81 cm, 82 cm, 83 cm, 84 cm, 85 cm, 86 cm, 87 cm, 88 cm, 89 cm, 90 cm, 91 cm, 92 cm, 93 cm, 94 cm, 95 cm, 96 cm, 97 cm, 98 cm, 99 cm, or 100 cm.

In one embodiment, the at least one tube has a diameter ranging from 0.1 cm to 3 cm.

In one embodiment, the at least one tube has a diameter of at least 0.1 cm, 0.2 cm, 0.3 cm, 0.4 cm, 0.5 cm, 0.6 cm, 0.7 cm, 0.8 cm, 0.9 cm, 1 cm, 1.1 cm, 1.2 cm, 1.3 cm, 1.4 cm, 1.5 cm, 1.6 cm, 1.7 cm, 1.8 cm, 1.9 cm, 2 cm, 2.1 cm, 2.2 cm, 2.3 cm, 2.4 cm, 2.5 cm, 2.6 cm, 2.7 cm, 2.8 cm, 2.9 cm, or 3 cm.

In one embodiment, the first and/or second supplying reservoir 21, 22 is sterile.

In one embodiment, the first and/or second supplying reservoir 21, 22 is disposable.

In one embodiment, the first and/or second supplying reservoir 21, 22 is reusable. In this embodiment, said supplying reservoir has to be cleaned following use.

In one embodiment, the first and/or second supplying reservoir 21, 22 is a chamber, a container, a bottle, a tube, or a pouch.

In one embodiment, the first and/or second supplying reservoir 21, 22 is fluidly connected to the device 1.

In one embodiment, the first and/or second supplying reservoir 21, 22 is clipped to the device 1.

In one embodiment, the collecting reservoir 23 is configured to collect a saline solution comprising purified mesenchymal stem cells.

In one embodiment, the collecting reservoir 23 is disposable.

In one embodiment, the collecting reservoir 23 is reusable. In this embodiment, said collecting reservoir 23 has to be cleaned following use.

In one embodiment, the collecting reservoir 23 is sterile.

In one embodiment, the collecting reservoir 23 is a chamber, a container, a bottle, a tube, or a pouch.

In one embodiment, the collecting reservoir 23 is configured to collect a solution or a suspension having a volume ranging from 20 ml to 400 ml, from 30 ml to 400 ml, from 40 ml to 400 ml, from 50 ml to 400 ml, from 100 ml to 400 ml, from 150 ml to 400 ml, from 200 ml to 400 ml, from 250 ml to 400 ml, from 300 ml to 400 ml, from 350 ml to 400 ml, from 20 ml to 350 ml, from 20 ml to 300 ml, from 20 ml to 250 ml, from 20 ml to 200 ml, from 20 ml to 150 ml, from 20 ml to 100 ml, or from 20 ml to 50 ml.

In one embodiment, the collecting reservoir 23 is fluidly connected to the at least one outlet 12 of the device 1 by clipping, gluing, at least one tube (as described hereabove) or any attachment means.

In one embodiment, the collecting reservoir 23 is configured to collect a first flow passing in the device 1 and/or a second flow passing in the device 1, wherein the second flow is a suspension of isolated and purified mesenchymal stem cells.

In one embodiment illustrated on FIG. 8, the system 2 comprises two collecting reservoir 231, 232: a first collecting reservoir 231 configured to collect the first flow passing in the device 1, and a second collecting reservoir 232 configured to collect the second flow passing in the device 1, i.e. a suspension of isolated and purified mesenchymal stem cells.

In one embodiment, the at least one pumping means 24 is configured to supply the device 1 with the at least one sample and/or the at least one saline solution.

In one embodiment, the system 2 further comprises two pumping means 24:
a first pumping means configured to supply the device 1 with the at least one sample, said first pumping means being fluidly connected with the first supplying reservoir 21; and
a second pumping means configured to supply said the device 1 with the at least one saline solution, said second pumping means being fluidly connected with the second supplying reservoir 22.

In one embodiment, the at least one pumping means 24 is a mechanical pumping means.

In one embodiment, the at least one pumping means 24 is a mechanical pump.

In one embodiment, the at least one pumping means 24 is a pump, a syringe pump, a peristaltic pump, or a piezoelectric type of pump.

In one embodiment, the system 2 comprises two flow controllers.

In one embodiment, the at least one flow controller 25 is a flow meter.

The flow controller 25 allows a fine control of the flow rate of the fluid passing through the device 1. Different flow rates can be chosen for different solutions. For example, the flow rate of a sample comprising mesenchymal stem cells may be slower than the flow of a saline solution used to rinse the surface 14 of the device 1 and collect the mesenchymal stem cells at the output of the device 1.

In one embodiment, the at least one flow controller 25 comprises at least one switch configured to switch on and off the flow of the fluid passing through the device 1.

In one embodiment, the at least one flow controller 25 comprises at least one output switch located at the at least one output of the device 1 configured to allow a fluid to pass from said device 1 to the collecting reservoir 23.

In one embodiment, the at least one flow controller 25 comprises at least one sensor configured to measure different flow rates. In one embodiment the at least one flow controller 25 comprises two sensors configured to measure different flow rates, one sensor being located at the input of the device 1 and one sensor being located at the output of the device 1.

In one embodiment, the at least one flow controller 25 comprises at least one bubble trap.

In one embodiment, the at least one flow controller 25 comprises at least one optical reader to display the measure of the flow.

In one embodiment, the device 1 comprises two inlets 11; the first supplying reservoir 21 comprises a first tubing configured to fluidly connect the first inlet to the first supplying reservoir 21 comprising a sample comprising mesenchymal stem cells; and the second supplying reservoir 22 comprises a second tubing configured to fluidly connect the second inlet to the second supplying reservoir 22 comprising a saline solution.

In one embodiment, the at least one flow controller 25 is configured to flow a fluid passing through the first tubing at a first flow rate and to flow a fluid passing through the second tubing at a second flow rate, wherein the first flow rate is slower than the second flow rate.

In one embodiment, the system 2 further comprises a control unit, said control unit comprises a software configured to control the flow rates. In this embodiment, the software may control the first flow rate, the second flow rate, or the pressure delivered by the at least one pumping means 24. In this embodiment, the method implemented by the system 2 may be semi-automated or automated. In this embodiment, the method implemented by the system 2 may be a computer implemented method.

In one embodiment, the system 2 further comprises an optical sensor configured to evaluate the quality of the suspension of purified mesenchymal stem cells.

In one embodiment, the system 2 further comprises a derivation tube located at or near the outlet 12 configured to fluidly connect the flowcytometer 26 to the device 1. Said derivation tube may pass through a reading chamber where the quantity of mesenchymal stem cells in the suspension of purified mesenchymal stem cells can be measured. In this embodiment, a tag or a cell marker can be added to said suspension of purified mesenchymal stem cells to tag mesenchymal stem cells, this step may be manual or automated.

In one embodiment, the at least one output switch of the at least one flow controller 25 is controlled by a software according to the evaluation performed by the optical sensor. In this embodiment, the output switch will allow a fluid to pass from the device 1 to at least one collecting reservoir 23 configured to collect the suspension of purified mesenchymal stem cells if the mesenchymal stem cells are purified and free of cellular debris.

In one embodiment, the system 2 can implement a method for isolating, retaining, separating and/or purifying adipose-derived mesenchymal stem cells as described hereafter.

In another aspect, illustrated in FIG. 6, this invention relates to a method for isolating, retaining, separating and/or purifying mesenchymal stem cells from a sample comprising mesenchymal stem cells.

Said method comprises the following steps:
pushing said sample in a device 1 as described hereabove at a first flow rate allowing to separate said sample into a first remaining sample comprising mesenchymal stem cells on the at least one surface 14 and into a second resulting solution being evacuated from the device 1;
evacuating the second resulting solution from the device 1;
pumping a saline solution in the device 1 at a second flow rate different from the first flow rate; and
collecting the saline solution comprising isolated and purified mesenchymal stem cells.

The first flow rate is slower than the second flow rate. The ratio between the first flow rate and the second flow rate is ranging from 2 to 50.

In another aspect, this invention relates to a method for isolating, retaining, separating and/or purifying mesenchymal stem cells from a heterogeneous population of cells comprising mesenchymal stem cells.

Said method comprises the following steps:
pushing said heterogeneous population of cells in a device (1) according to any one of claims 1 to 7, at a first flow rate allowing to separate said heterogeneous population of cells into a first remaining sample comprising mesenchymal stem cells on the at least one surface (14) and into a second resulting solution being evacuated from the device (1);
evacuating the second resulting solution from the device (1);
pumping a saline solution in the device (1) at a second flow rate different from the first flow rate; and
collecting the saline solution comprising isolated, retained, separated and/or purified mesenchymal stem cells;
wherein the first flow rate is slower than the second flow rate;
wherein the ratio between the first flow rate and the second flow rate is ranging from 2 to 50.

In one embodiment, the sample is pushed in the device 1 using pumping means 24.

In one embodiment, the first flow rate is ranging from 10 to 150 ml/min.

In one embodiment, the second flow rate is ranging from 100 to 500 ml/min.

Said method is based on the adherence properties of mesenchymal stem cells on polymer materials. By pumping a sample on a surface having an affinity for mesenchymal stem cells, the mesenchymal stem cells in said sample decelerate while other cells and particles are carried away allowing to separate the sample into a first remaining sample comprising mesenchymal stem cells located at the surface vicinity and into a second resulting solution being evacuated from the device 1. Said second resulting solution comprises other cells, particles, or cellular debris.

According to the Applicant, by advantageously selecting the first flow rate, the material of the surface, the mesenchymal stem cells do not adhere on the surface but only slow down due to their natural attraction to the surface. Thus, stem cells are retained due to their attraction to the surface while other components of the first flow (cellular debris, extracellular matrix product, pre-adipocytes . . . ) are drained away at a rate equal to the first flow rate. However, it is important that the mesenchymal stem cells do not adhere on the surface so that they are not attached to it; this prevents the risk of differentiation of the stem cells in the device 1. The length of the surface, i.e. the length of the route that the fluid has to take, may also be of importance as the longer the surface is, the more the stem cells may be retained.

The sample of mesenchymal stem cells has to be pumped in the device 1 to ensure a sufficient flow rate. Using only the actual gravity of the sample is not sufficient and do not allow a fine control on the flow rate of said sample in the device 1. In addition gravity will result in significant differences in flow speed of the sample into the device and inconsistent segregation of the mesenchymal stem cells between two different samples. Furthermore gravity may allow cellular and extra cellular matrix tissue debris to attach to the mesenchymal stem cells and the device's surface.

The first flow rate is configured to be fast enough so that the mesenchymal stem cells do not adhere to the surface, but slow enough so that said mesenchymal stem cells decelerate in the vicinity of the surface. The advantage is that the mesenchymal stem cells are retained for a short time on the surface, this time being short enough to prevent the differentiation of the stem cells, while the other cells or cellular debris are evacuated from the device 1. Thus, the differentiated flow of the mesenchymal stem cells compared to other cells or cellular debris comprised in the sample allows for the isolation, retention, separation and/or purification of said mesenchymal stem cells.

The second flow rate is configured to be faster than the first flow rate, and fast enough so that the mesenchymal stem cells are carried away with said second flow.

The purified mesenchymal stem cells may be delivered immediately to different specialists for the treatment of a variety of conditions, for instance in orthopedics (joint diseases, bone grafts), ophthalmology (macular degeneration, glaucoma), uro-gynecology (urinary incontinence, erectile dysfunction), plastic surgery (face peels, hair regrowth, scars, lipofilling, lipostructure, breast augmentation), wound healing, salivary glands stimulation after radiotherapy, peripheral arterial disease. The purified mesenchymal stem cells may be reinjected in the subject who provided the sample of adipose tissue. They may be injected in joints, salivary glands, retina, skin, muscle, or mixed with bone graft. These cells may be used for any tissue regeneration procedure or method. They may also be topically applied on wounds, burn wounds, ulcers, after chemical or mechanical face peeling.

The saline solution comprising purified mesenchymal stem cells is also referred hereafter as a suspension of purified mesenchymal stem cells.

In one embodiment, the method is chemical-free and enzyme-free. In this embodiment, the method is compliant with the requirements of Regulation (EC) no 1394/2007 of the European Parliament and of the Council of 13 Nov. 2007 on advanced therapy medicinal products. This embodiment prevents a chemical modification or pollution of the stem cells.

In one embodiment, the method is performed with a system 2 as described hereabove.

In one embodiment, the method is performed in less than 30 minutes. This method is very fast and allows the use the purified stem cells in clinical application in situ: the sample of adipose tissue can be provided and the resulting stem cells can be purified during the same clinical procedure.

In one embodiment, the sample is pushed in the device 1 at a first flow rate using a syringe connected at the at least one inlet of said device 1, allowing to separate said sample into a first remaining sample comprising mesenchymal stem cells on the at least one surface 14 and into a second resulting solution being evacuated from the device 1; then a saline solution is pushed in the device at a second stronger flow rate to collect isolated, retained, separated and/or purified mesenchymal stem cells.

In one embodiment, the sample is pushed in the device 1, the device being a rectangular parallelepiped, at a first flow rate using a syringe connected at the at least one inlet of said device 1, allowing to separate said sample into a first remaining sample comprising mesenchymal stem cells on the at least one surface 14 and into a second resulting solution being evacuated from the device 1; then a saline solution is pushed in the device at a second stronger flow rate to collect isolated and purified mesenchymal stem cells.

In one embodiment, a centrifugal force is applied on the device 1 while the sample is pushed through said device 1 and/or while the sample is in the device 1. This embodiment is particularly advantageous as it increases closeness and/or contact between mesenchymal stem cells and the surface 14.

In one embodiment, the method is performed in less than 30 minutes, 25 minutes, 20 minutes, 15 minutes, 10 minutes, or 5 minutes.

In one embodiment, the method is manual.

In one embodiment, the method is semi-automated.

In one embodiment, the method is automated. In this embodiment, a control unit is used to control parameters like the flow rates.

In one embodiment, during the method, i.e. during its passing through the device 1, the temperature of the sample is adjusted between 10 and 37° C.

In one embodiment, the mesenchymal stem cells do not differentiate during the method.

In one embodiment, the sample is a sample of adipose tissue, bone marrow, blood umbilical cord, molar, amniotic fluid, follicular tissue (hair), or a sample from a human embryo obtained without the destruction of an embryo.

In one embodiment, the sample has been frozen prior introduction in the device 1. In this embodiment, the device 1 allows the purification, retention, separation and/or isolation of mesenchymal stem cells from a frozen sample.

In one embodiment, the sample is a lipoaspirate.

In one embodiment, the method further comprises a step of providing a sample comprising adipose tissue, bone marrow, blood umbilical cord, molar, amniotic fluid, follicular tissue (hair), or a sample from a human embryo obtained without the destruction of an embryo. In this embodiment, the sample is provided prior pumping said sample in the device 1.

In one embodiment, the sample of adipose tissue is obtained by a minimally invasive procedure such as lipoaspiration, or minimally invasive surgery. In this embodiment, surgery may comprise incision and dissection.

In one embodiment, the sample of adipose tissue, bone marrow, blood umbilical cord, molar, amniotic fluid, follicular tissue (hair), or a sample from a human embryo obtained without the destruction of an embryo, is obtained by any means known by the person skilled in the art.

In one embodiment, the sample has a volume ranging from 20 cm³ to 400 cm³, from 20 cm³ to 200 cm³, preferably from 20 cm³ to 100 cm³, more preferably from 40 cm³ to 60 cm³.

In one embodiment, the sample has a volume of at least 20 cm³, 30 cm³, 40 cm³, 50 cm³, 60 cm³, 70 cm³, 80 cm³, 90 cm³, 100 cm³, 110 cm³, 120 cm³, 130 cm³, 140 cm³, 150 cm³, 160 cm³, 170 cm³, 180 cm³, 190 cm³, or 200 cm³.

In one embodiment, the sample has a volume ranging from 20 ml to 400 ml, from 20 ml to 200 ml, preferably from 20 ml to 100 ml, more preferably from 40 ml to 60 ml.

In one embodiment, the sample has a volume of at least 20 ml, 30 ml, 40 ml, 50 ml, 60 ml, 70 ml, 80 ml, 90 ml, 100 ml, 110 ml, 120 ml, 130 ml, 140 ml, 150 ml, 160 ml, 170 ml, 180 ml, 190 ml, or 200 ml.

In one embodiment, the cell suspension collected from a subject can be divided in several samples, each sample having a predefined volume.

In one embodiment, the sample comprises unpurified mesenchymal stem cells. In this embodiment, the sample may comprise mesenchymal stem cells, cellular debris, extracellular matrix product, pre-adipocytes, leucocytes, endothelial cells, smooth muscle cells, pericytes, fibroblasts, erythrocytes, B and T cells, macrophages, monocytes, or mast cells.

In one embodiment, the first flow rate is at least 10 ml/min, 11 ml/min, 12 ml/min, 13 ml/min, 14 ml/min, 15 ml/min, 16 ml/min, 17 ml/min, 18 ml/min, 19 ml/min, 20 ml/min, 21 ml/min, 22 ml/min, 23 ml/min, 24 ml/min, 25 ml/min, 26 ml/min, 27 ml/min, 28 ml/min, 29 ml/min, 30 ml/min, 31 ml/min, 32 ml/min, 33 ml/min, 34 ml/min, 34 ml/min, 36 ml/min, 37 ml/min, 38 ml/min, 39 ml/min, 40 ml/min, 41 ml/min, 42 ml/min, 43 ml/min, 44 ml/min, 45 ml/min, 46 ml/min, 47 ml/min, 48 ml/min, 49 ml/min, 50 ml/min, 51 ml/min, 52 ml/min, 53 ml/min, 54 ml/min, 55 ml/min, 56 ml/min, 57 ml/min, 58 ml/min, 59 ml/min, 60 ml/min, 61 ml/min, 62 ml/min, 63 ml/min, 64 ml/min, 65 ml/min, 66 ml/min, 67 ml/min, 68 ml/min, 69 ml/min, 70 ml/min, 71 ml/min, 72 ml/min, 73 ml/min, 74 ml/min, 75 ml/min, 76 ml/min, 77 ml/min, 78 ml/min, 79 ml/min, 80 ml/min, 81 ml/min, 82 ml/min, 83 ml/min, 84 ml/min, 85 ml/min, 86 ml/min, 87 ml/min, 88 ml/min, 89 ml/min, 90 ml/min, 91 ml/min, 92 ml/min, 93 ml/min, 94 ml/min, 95 ml/min, 96 ml/min, 97 ml/min, 98 ml/min, 99 ml/min, 100 ml/min, 110 ml/min, 120 ml/min, 130 ml/min, 140 ml/min, or 150 ml/min.

In one embodiment, the second flow rate is at least 100 ml/min, 110 ml/min, 120 ml/min, 130 ml/min, 140 ml/min, 150 ml/min, 160 ml/min, 170 ml/min, 180 ml/min, 190 ml/min, 200 ml/min, 210 ml/min, 220 ml/min, 230 ml/min, 240 ml/min, 250 ml/min, 260 ml/min, 270 ml/min, 280 ml/min, 290 ml/min, 300 ml/min, 310 ml/min, 320 ml/min, 330 ml/min, 340 ml/min, 350 ml/min, 360 ml/min, 370 ml/min, 380 ml/min, 390 ml/min, 400 ml/min, 410 ml/min, 420 ml/min, 430 ml/min, 440 ml/min, 450 ml/min, 460 ml/min, 470 ml/min, 480 ml/min, 490 ml/min, or 500 ml/min.

In one embodiment, the ratio between the first flow rate and the second flow rate is at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

In one embodiment, the second resulting solution is collected and/or eliminated after evacuation from the device 1.

In one embodiment, the second resulting solution is collected in a collecting reservoir 23, i.e. a chamber, a container, a bottle, a tube, or a pouch.

In one embodiment, the saline solution comprises NaCl in water at a level ranging from 0.20 to 0.90% w/v.

In one embodiment, the saline solution comprises NaCl in water at a level ranging from 0.20 to 0.90‰.

In one embodiment, the saline solution comprises 0.90% w/v of NaCl in water.

In one embodiment, the saline solution comprises 0.90‰ of NaCl in water.

In one embodiment, the saline solution comprises 0.45% w/v of NaCl in water.

In one embodiment, the saline solution comprises 0.45‰ of NaCl in water.

In one embodiment, the purified mesenchymal stem cells are collected in a collecting reservoir 23, i.e. a chamber, a container, a pipette, by gently scrubbing the surface 14 and/or by gently agitating the surface 14 or the device 1 in a solution.

In one embodiment, the purified mesenchymal stem cells may comprise leucocytes.

In one embodiment, the purified mesenchymal stem cells may comprise leucocytes at a level ranging from 1 to 1000% of the total purified mesenchymal stem cells volume.

In one embodiment, the purified mesenchymal stem cells do not comprise leucocytes. This embodiment is especially advantageous for knee, hip joint, or eye treatment.

In one embodiment, the purified mesenchymal stem cells do not comprise cellular debris, extracellular matrix product, pre-adipocytes, leucocytes, endothelial cells, smooth muscle cells, pericytes, fibroblasts, erythrocytes, B and T cells, macrophages, monocytes, or mast cells. Cellular debris could indeed trigger an inflammatory reaction. This embodiment allows the injection of said purified mesenchymal stem cells in a subject.

In one embodiment, the purified mesenchymal stem cells are ready to be used.

In one embodiment, the saline solution is pumped in the device 1 through an inlet of said device 1.

In one embodiment, the saline solution is pumped in the device 1 through an outlet of said device 1. This embodiment is advantageous as it allows a better collection of the isolated, separated, retained and/or purified cells.

In one embodiment, method comprises the following steps:
pushing said sample in a device 1 as described hereabove through an inlet of said device at a first flow rate allowing to separate said sample into a first remaining sample comprising mesenchymal stem cells on the at least one surface 14 and into a second resulting solution being evacuated from the device 1;
evacuating the second resulting solution from the device 1;
pumping a saline solution in the device 1 through an inlet of said device 1 at a second flow rate different from the first flow rate; and
collecting the saline solution comprising isolated, retained, separated and/or purified mesenchymal stem cells.

In one embodiment, method comprises the following steps:
pushing said sample in a device 1 as described hereabove through an inlet of said device at a first flow rate allowing to separate said sample into a first remaining sample comprising mesenchymal stem cells on the at least one surface 14 and into a second resulting solution being evacuated from the device 1;
evacuating the second resulting solution from the device 1;
pumping a saline solution in the device 1 through an outlet of said device 1 at a second flow rate different from the first flow rate; and
collecting the saline solution comprising isolated, retained, separated and/or purified mesenchymal stem cells.

In one embodiment, the method comprises the following steps:
a) pushing a heterogeneous population of cells in a device 1 as described hereabove at a first flow rate allowing to separate said sample into a first remaining sample comprising mesenchymal stem cells on the at least one surface 14 and into a second resulting solution being evacuated from the device 1;
b) evacuating the second resulting solution from the device 1;
c) pumping a saline solution in the device 1 at a second flow rate different from the first flow rate; and
d) collecting the saline solution comprising the first remaining sample comprising mesenchymal stem cells;
e) pushing said saline solution comprising the first remaining sample comprising mesenchymal stem cells in the device at a flow rate allowing to separate said saline solution into a remaining sample comprising mesenchymal stem cells on the at least one surface 14 and into a resulting solution being evacuated from the device 1;
f) evacuating the resulting solution from the device 1;
g) pumping a saline solution in the device 1 at a flow rate different from the precedent flow rate; and
h) collecting the saline solution comprising the remaining sample comprising mesenchymal stem cells;
   wherein steps e) to h) are repeated several times before collecting a homogeneous population of mesenchymal stem cells.

This embodiment is particularly advantageous as is allows a further isolation, retention, separation and/or purification of the mesenchymal stem cells, as each time steps e) to h) are repeated, more unwanted cells or particles are removed from the suspension comprising said stem cells.

In one embodiment, steps e) to h) are repeated once, twice, 3 times, 4 times, 5 times, 6 times, 7 times, 8 times, 9 times or 10 times. This embodiment is particularly advantageous as it improves the purification with each repetition of steps e) to h).

In one embodiment, the method comprises 1, 2, 3, 4, 5, 6, 7, 8, 9 or 10 cycles of steps e) to h). One cycle of steps e) to h) refers to one occurrence of said steps in the method. This embodiment is particularly advantageous as it improves the purification with each cycle.

In one embodiment, steps e) to h) are repeated in the same device 1.

In one embodiment, each cycle of steps e) to h) are repeated in a different device 1. In this embodiment, several devices 1 can be placed in a series to further isolate, retain, separate and/or purify the population of cells.

In one embodiment, the second resulting solution may be evacuated from the device 1 through the inlet 11 or the outlet 12.

In one embodiment, the method further comprises a centrifugation step before pumping the sample in the device 1.

In one embodiment, the method further comprises the following steps before pumping the sample in the device 1:
subjecting the sample to at least one centrifugation round;
collecting the centrifuged layer comprising mesenchymal stem cells; said centrifuged layer will be pumped in the device 1 according to the method of the invention.

The aim of the centrifugation step is to isolate mesenchymal stem cells prior purification in the device 1. Thus, the step of isolation of said mesenchymal stem cells is mechanical and do not use any chemicals or enzymes. This embodiment prevents a chemical modification or pollution of the stem cells.

According to one embodiment, the concentration of cells in an isotonic solution is variable.

According to one embodiment, the concentration of the saline solution is variable.

According to one embodiment, the sample comprising mesenchymal stem cells is beforehand mixed with a saline solution and/or water at a temperature between 25° C. and 41° C. Adding water to the sample allows to separate cells from tissue via osmotic shock (or osmotic stress). In this embodiment, said osmotic shock has to be made carefully to avoid the differentiation of the stem cells due to a strong traumatism.

According to one embodiment, the sample comprising mesenchymal stem cells is beforehand mixed with a saline solution and/or water at a temperature of at least 25° C., 26° C., 27° C., 28° C., 29° C., 30° C., 31° C., 32° C., 33° C., 34° C., 35° C., 36° C., 37° C., 38° C., 39° C., 40° C., or 41° C.

According to one embodiment, saline solution and water are used in same quantity.

According to one embodiment, after mixing the sample of adipose tissue with a saline solution and/or water, said sample is heated and agitated.

In one embodiment, said sample is heated and agitated at a temperature ranging from 25° C. to 41° C. for at least 5 minutes.

In one embodiment, said sample is heated and agitated at a temperature of at least 25° C., 26° C., 27° C., 28° C., 29° C., 30° C., 31° C., 32° C., 33° C., 34° C., 35° C., 36° C., 37° C., 38° C., 39° C., 40° C., or 41° C. for at least 5 minutes.

In a preferred embodiment, said sample is heated and agitated at 39° C. for 10 minutes.

In one embodiment, said sample is heated and agitated in a vortex heating chamber.

In one embodiment, the step of subjecting the sample to at least one centrifugation round comprises the following steps:
subjecting the sample to a first centrifugation round;
collecting the intermediate centrifuged layer;
subjecting said intermediate centrifuged layer to a second centrifugation round; and
collecting the superficial centrifuged layer comprising mesenchymal stem cells; said layer is then pumped in the device 1 according to the method of the present invention.

After the first round of centrifugation, the sample will show a plurality of layers: a superficial layer of oil and fat tissue, an intermediate yellowish layer and a bottom pellet containing the red blood cells, fibroblasts, smooth muscle cells, and/or white blood cells. The intermediate centrifuged layer comprises the mesenchymal stem cells and will be collected to be subjected to a second centrifugation round.

After the second of centrifugation, the sample comprises two layers of suspension. The superficial centrifuged layer comprises the stem cells and is collected to be subjected to the purification steps.

Thus, the two centrifugation rounds allow the separation of the stem cells from the other populations.

In one embodiment, the at least one centrifugation round is performed at a rate below 1500 rounds per minute.

In one embodiment, the at least one centrifugation round is performed at a rate below 1500 rounds per minute, 1400 rounds per minute, 1300 rounds per minute, 1200 rounds per minute, 1100 rounds per minute, 1000 rounds per minute, 900 rounds per minute, 800 rounds per minute, 700 rounds per minute, 600 rounds per minute, or 500 rounds per minute.

In one embodiment, the at least one centrifugation round is performed for a time ranging from 3 to 20 min.

In one embodiment, the at least one centrifugation round is performed for at least 3 min, 4 min, 5 min, 6 min, 7 min, 8 min, 9 min, 10 min, 11 min, 12 min, 13 min, 14 min, 15 min, 16 min, 17 min, 18 min, 19 min, or 20 min.

In one embodiment, the first and the second centrifugation rounds are preferably performed at a rate below 1500 rounds per minute. It is known that a strong centrifugation, over 1500 rounds per minute will precipitate the stem cells with the other cells in a pellet at the bottom of the tubes.

In one embodiment, the first and the second centrifugation rounds are performed at a rate below 1500 rounds per minute, 1400 rounds per minute, 1300 rounds per minute, 1200 rounds per minute, 1100 rounds per minute, 1000 rounds per minute, 900 rounds per minute, 800 rounds per minute, 700 rounds per minute, 600 rounds per minute, or 500 rounds per minute.

In one embodiment, the first and the second centrifugation rounds are preferably performed at a different rate; the first centrifugation round is performed at a higher rate than the second centrifugation round.

In one embodiment, the collected intermediate centrifuged layer is resuspended in saline solution before the second centrifugation round.

In one embodiment, the intermediate centrifuged layer and/or the superficial centrifuged layer comprising mesenchymal stem cells are collected using a pipette, or any means known by the person skilled in the art. In this embodiment, the pipette is sterile.

The method does not comprise a sonication step of the sample, i.e. the sample is not subjected to ultrasounds.

In one embodiment, the method further comprises a measuring step to measure the quantity and/or the concentration of mesenchymal stem cells in the suspension of purified mesenchymal stem cells using a flowcytometer 26 as described hereabove. This step will allow a personalized and more efficient use of the resulting suspension of purified mesenchymal stem cells. Indeed, this may lead the user to the decision to take an additional sample to have more cells available and reach a therapeutic threshold.

In one embodiment, the measuring step may be manual, semi-automated, or automated.

In another aspect, the invention relates to the use of the device 1 and/or the system 2 as described hereabove for isolating, retaining, separating, and/or purifying adipose-derived mesenchymal stem cells, bone marrow-derived stem cells, blood-derived mesenchymal stem cells, blood umbilical cord stem cells, molar stem cells, amniotic fluid stem cells, follicular stem cells, or human embryonic stem cells (hESC) obtained without the destruction of an embryo.

The purified mesenchymal stem cells may be used for the treatment of a variety of conditions, for example in orthopedics (joint diseases, bone grafts), ophthalmology (macular degeneration, glaucoma), uro-gynecology (urinary incontinence, erectile dysfunction), plastic surgery (face peels, hair regrowth, scars, lipofilling, lipostructure, breast augmentation), wound healing, salivary glands stimulation after radiotherapy, peripheral arterial disease. The purified mesenchymal stem cells may be reinjected in the subject that provided the sample of adipose tissue. They may be injected in joints, salivary glands, retina, skin, muscle, or mixed with bone graft. These cells may be used for any tissue regeneration procedure or method. They may also be topically applied on wounds, burn wounds, ulcers, after chemical or mechanical face peeling.

While various embodiments have been described and illustrated, the detailed description is not to be construed as being limited hereto. Various modifications can be made to the embodiments by those skilled in the art without departing from the true spirit and scope of the disclosure as defined by the claims.

### BRIEF DESCRIPTION OF THE DRAWINGS

FIG. 1 is a device 1 according to the invention.

FIG. 2 is a device 1 according to the invention.

FIG. 3 is a device 1 according to the invention.

FIG. 4 is a device 1 according to the invention.

FIG. 5 is a device 1 according to the invention, said device 1 having a planar spiral geometry.

FIG. 6 is a histogram showing the method for isolating and purifying adipose-derived mesenchymal stem cells from a sample according to the invention.

FIG. 7 is a system 2 according to the invention.

FIG. 8 is a system 2 according to the invention, comprising a first collecting reservoir 231 and a second collecting reservoir 232.

FIG. 9 is a system 2 according to the invention, comprising a first collecting reservoir 231, a second collecting reservoir 232 and a flowcytometer 26.

FIG. 10A is a device 1 according to the invention, said device being configured to rotate.

FIG. 10B is a device 1 according to the invention, said device being configured to rotate.

FIG. 11A illustrates the flow of a cell suspension in a device 1, wherein said device does not rotate.

FIG. 11B illustrates a cell in a cell suspension in a device 1, wherein said device does not rotate.

FIG. 11C illustrates the flow of a cell suspension in a device 1, wherein said device rotates.

FIG. 11D illustrates a cell in a cell suspension in a device 1, wherein said device rotates. Said cell is much closer to the surface 14 than the cell in the non-rotating device 1.

FIG. 12 is a device 1 according to the invention, said device 1 having a parallelepiped geometry. Even though, the device is represented with a rectangular section, it may also have a square, oval, or circular section.

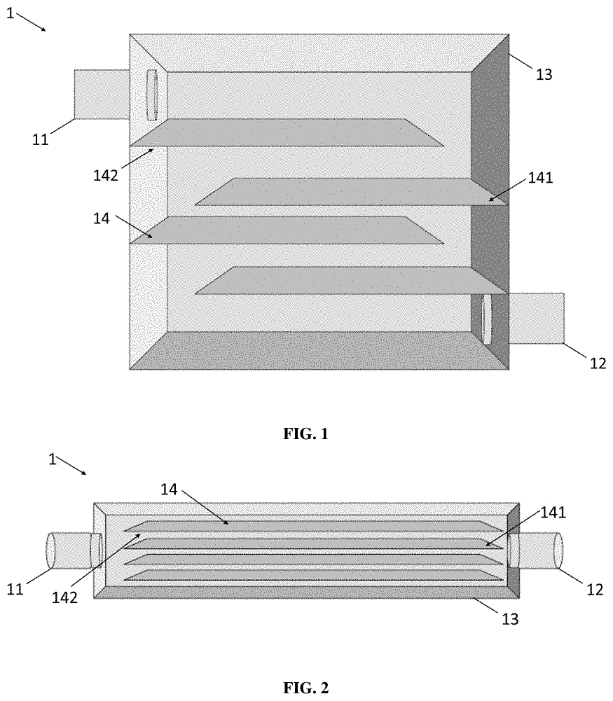

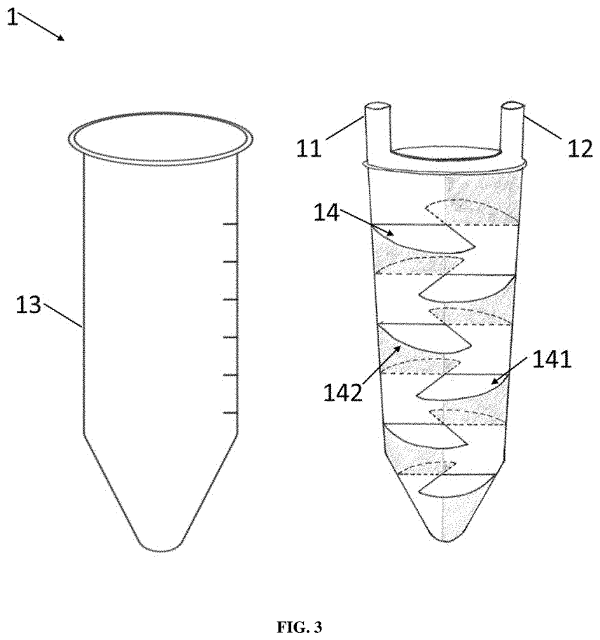

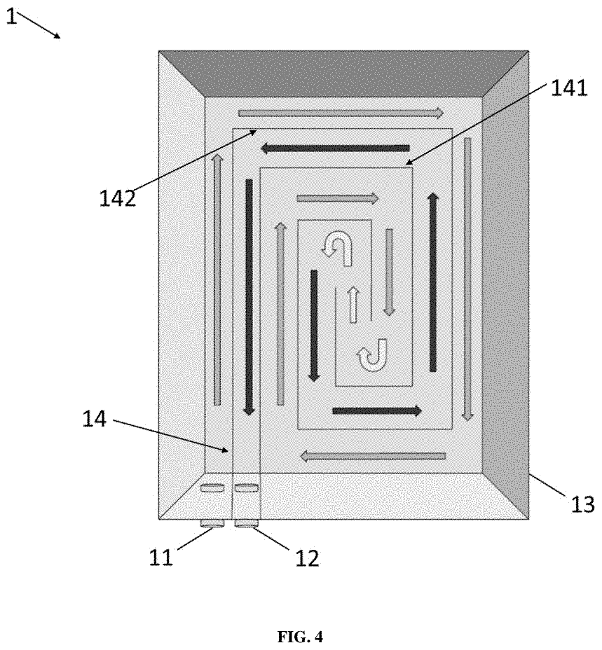

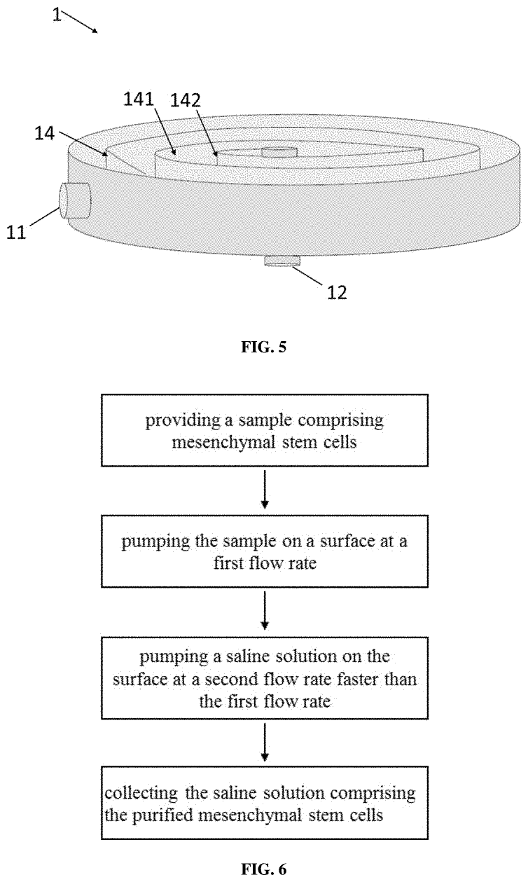

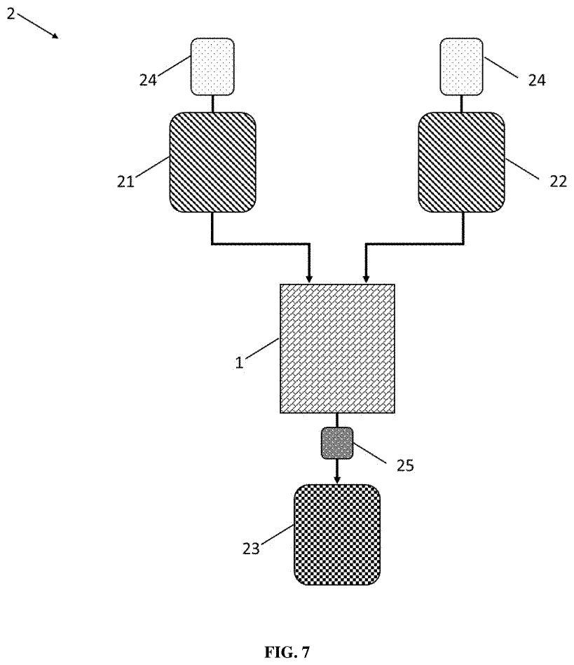

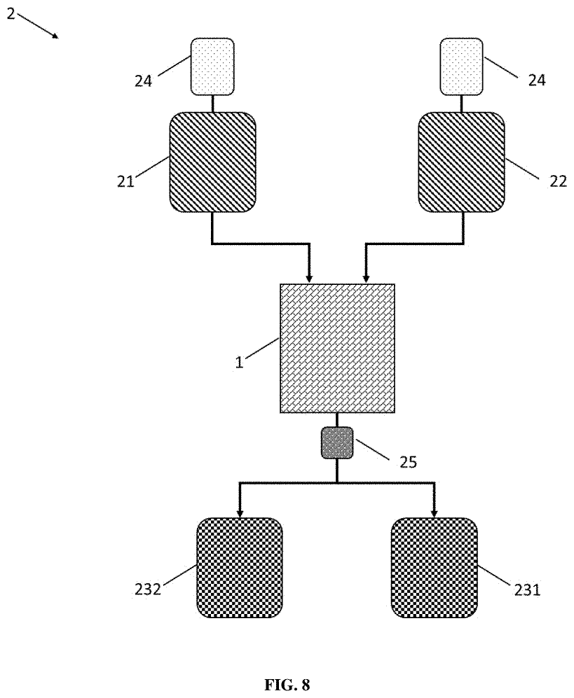

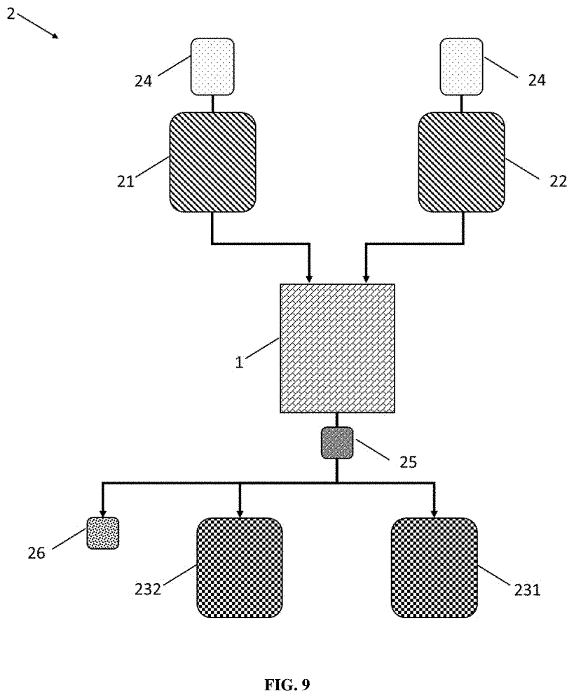

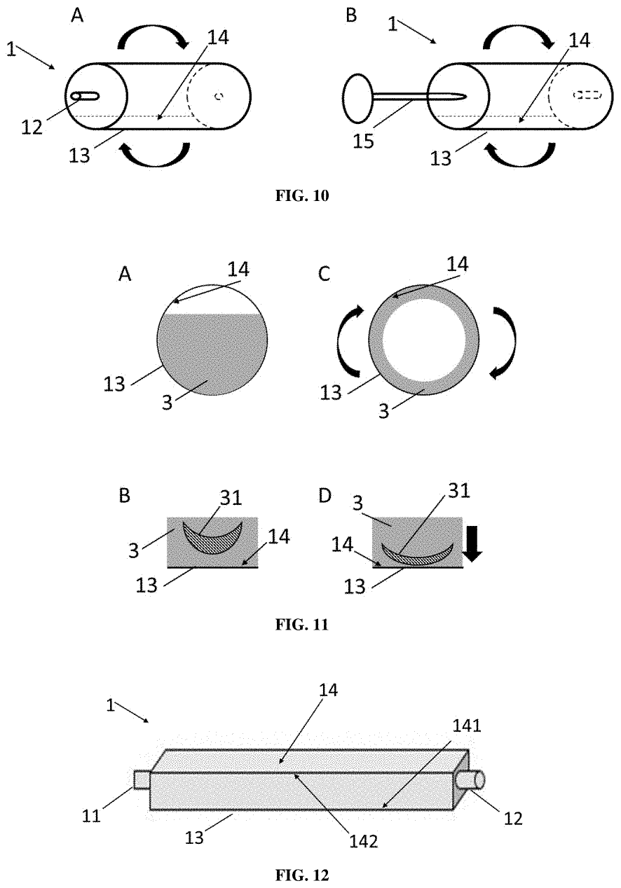

### EXAMPLES

The present invention is further illustrated by the following examples.

### Example 1

### Materials and Methods

### Material:

sample of adipose tissue;
device 1 according to the invention.

### Methods

Adipose tissue is collected by lipoaspiration. The fat tissue is added with an equal volume of saline and an equal volume of non-pyrogenic distilled water and introduced into a vortex heating chamber for 10 minutes at 39° C. The resulting suspension is centrifuged at 1200 rpm for 10 minutes. After the centrifugation round, there are a superficial layer of oil and fat tissue, an intermediate yellowish layer and a bottom pellet containing the red blood cells, fibroblasts, smooth muscle cells, white blood cells. The intermediate centrifuged layer is collected with a sterile pipette and resuspended with saline. A second centrifugation is performed at 700 rpm for 5 minutes. This results in two layers. The superficial layer containing the stem cells is collected and ready for purification. These two centrifugations allow the separation of the stem cells from the other populations.

The resulting suspension is purified using the device 1 of the invention. The suspension is flowed at a first flow rate of 100 ml/mn in the device 1. Then, a rapid flush of 300 ml/mn with saline washes out the almost adherent cells, which are collected at the end of the device 1.

### Results

This results in a suspension of purified mesenchymal stem cells.

The same method was performed with a sample of bone marrow, blood umbilical cord, molar, amniotic fluid, follicular tissue (hair), or a sample from a human embryo obtained without the destruction of an embryo.

### Example 2

### Materials and Methods

### Material:

sample of adipose tissue;
device 1 according to the invention.

### Methods

Adipose tissue is collected surgically during a surgical intervention of lipofilling. The fat tissue is added with an equal volume of saline and an equal volume of non pyrogenic distilled water and introduced into a vortex heating chamber for 15 minutes at 39° C. The resulting suspension is centrifuged at 1000 rpm for 14 minutes. After the centrifugation round, there are a superficial layer of oil and fat tissue, an intermediate yellowish layer and a bottom pellet containing the red blood cells, fibroblasts, smooth muscle cells, white blood cells. The intermediate centrifuged layer is collected with a sterile pipette and resuspended with saline. A second centrifugation is performed at 700 rpm for 5 minutes. This results in two layers. The superficial layer containing the stem cells is collected and ready for purification. These two centrifugations allow the separation of the stem cells from the other populations.

The resulting suspension is purified using the device 1 of the invention. The suspension is flowed at a first flow rate of 100 ml/mn in the device 1. Then, a rapid flush of 300 ml/mn with saline washes out the almost adherent cells, which are collected at the end of the device 1.

### Results

This results in a suspension of purified mesenchymal stem cells.

The same method was performed with a sample of bone marrow, blood umbilical cord, molar, amniotic fluid, follicular tissue (hair), or a sample from a human embryo obtained without the destruction of an embryo.

### Example 3

### Materials and Methods

### Material:

sample of adipose tissue;
device 1 according to the invention.

### Methods

Adipose tissue is collected surgically during a surgical intervention of debridement of a chronic wound. The fat tissue is added with an equal volume of saline and an equal volume of non pyrogenic distilled water and introduced into a vortex heating chamber for 10 minutes at 39° C. The resulting suspension is centrifuged at 1200 rpm for 10 minutes. After the centrifugation round, there are a superficial layer of oil and fat tissue, an intermediate yellowish layer and a bottom pellet containing the red blood cells, fibroblasts, smooth muscle cells, white blood cells. The intermediate centrifuged layer is collected with a sterile pipette and resuspended with saline. A second centrifugation is performed at 700 rpm for 5 minutes. This results in two layers. The superficial layer containing the stem cells is collected and ready for purification. These two centrifugations allow the separation of the stem cells from the other populations.

The resulting suspension is purified using the device 1 of the invention. The suspension is flowed at a first flow rate of 200 ml/mn in the device 1. Then, a rapid flush of 400 ml/mn with saline washes out the almost adherent cells, which are collected at the end of the device 1.

### Results

This results in a suspension of purified mesenchymal stem cells.

The quantity of mesenchymal stem cells in the purified suspension of mesenchymal stem cells is measured using a flowcytometer 26.

The same method was performed with a sample of bone marrow, blood umbilical cord, molar, amniotic fluid, follicular tissue (hair), or a sample from a human embryo obtained without the destruction of an embryo.

### REFERENCES

1—Device for purifying mesenchymal stem cells;
11—Inlet;
12—Outlet;
13—Housing;
14—Surface;
141—Portion of surface;
142—Portion of surface;
15—rotating mechanism;
2—System;
21—First supplying reservoir;
22—Second supplying reservoir;
23—Collecting reservoir;
231—First collecting reservoir;
232—Second collecting reservoir;
24—Pumping means;
25—Flow controller;
26—Flowcytometer;
3—Flow
31—cell.

1-15. (canceled)

16. A device for isolating, retaining, separating and/or purifying mesenchymal stem cells from a heterogeneous population of cells comprising:
at least one inlet;
at least one outlet;
a housing;
at least one surface located in the housing, said surface having an affinity for mesenchymal stem cells;
wherein said surface is in communication with the inlet and the outlet; and
wherein the at least one surface comprises at least two portions facing each other, and separated by a distance ranging from 1 mm to 1 cm.

17. The device according to claim 16, wherein the at least one surface comprises a material selected in the group of polymeric material such as polystyrene, (PS), polyamine, polycarbonate (PC), poly-D-lysine (PDL), polycaprolactone, or inorganic material such as glass.

18. The device according to claim 16, wherein the at least one surface is smooth.

19. The device according to claim 16, wherein the at least two portions are slides or coverslips.

20. The device according to claim 16, wherein the at least two portions are facing each other in staggered rows.

21. The device according to claim 16, wherein the at least two portions are parallel to each other, or describe an angle to each other ranging from 0° to 30°.

22. The device according to claim 16, wherein the at least one surface is a planar spiral.

23. A method for isolating, retaining, separating and/or purifying mesenchymal stem cells from a heterogeneous population of cells comprising mesenchymal stem cells, comprising the following steps:
pushing said heterogeneous population of cells in a device according to claim 16, at a first flow rate allowing to separate said heterogeneous population of cells into a first remaining sample comprising mesenchymal stem cells on the at least one surface and into a second resulting solution being evacuated from the device;
evacuating the second resulting solution from the device;
pumping a saline solution in the device at a second flow rate different from the first flow rate; and
collecting the saline solution comprising isolated, retained, separated and/or purified mesenchymal stem cells;
wherein the first flow rate is slower than the second flow rate;
wherein the ratio between the first flow rate and the second flow rate is ranging from 2 to 50.

24. The method for isolating, retaining, separating and/or purifying mesenchymal stem cells from a heterogeneous population of cells according to claim 23, further comprising a centrifugation step before pumping the sample in the device.

25. The method for isolating, retaining, separating and/or purifying mesenchymal stem cells from a heterogeneous population of cells according to claim 23, wherein the first flow rate is ranging from 10 to 150 ml/min.

26. The method for isolating, retaining, separating and/or purifying mesenchymal stem cells from a heterogeneous population of cells according to claim 23, wherein the second flow rate is ranging from 100 to 500 ml/min.

27. A method for isolating, retaining, separating, and/or purifying mesenchymal stem cells selected from the group consisting of adipose-derived mesenchymal stem cells, bone marrow-derived mesenchymal stem cells, blood-derived mesenchymal stem cells, blood umbilical cord stem cells, molar stem cells, amniotic fluid stem cells, follicular stem cells, and human embryonic stem cells (hESC) obtained without the destruction of an embryo, comprising pushing a heterogeneous population of cells in a device according to claim 16, at a first flow rate allowing to separate said heterogeneous population of cells into a first remaining sample comprising said mesenchymal stem cells on the at least one surface of the device and into a second resulting solution being evacuated from the device.

28. A system for isolation, retention, separation and/or purification of mesenchymal stem cells from at least one heterogeneous population of cells, comprising:
a device according to claim 16;
a first supplying reservoir fluidly connected to at least one inlet of the device and configured to comprise at least one heterogeneous population of cells comprising mesenchymal stem cells;
a second supplying reservoir fluidly connected to at least one inlet of the device and configured to comprise at least one saline solution;
at least one collecting reservoir fluidly connected to at least one outlet of the device;
at least one pumping means fluidly connected to at least one supplying reservoir; and
at least one flow controller configured to control the flow rate of a fluid pumped through the inlet of the device.

29. The system for isolation, retention, separation and/or purification of mesenchymal stem cells according to claim 28, further comprising a flowcytometer.

30. The system for isolation, retention, separation and/or purification of mesenchymal stem cells according to claim 28, further comprising a temperature controller.
